(19) United States
(12) Patent Application Publication
(10) Pub. No.: US 2002/0058894 A1
(43) Pub. Date: May 16, 2002

(54) Arm support

(76) Inventor: Daniel J. Robbins, Santa Monica, CA (US)

Correspondence Address: OPPENHEIMER WOLFF & DONNELLY LLP, 38TH FLOOR, 2029 CENTURY PARK EAST, LOS ANGELES, CA, 90067-3024, US

(21) Appl. No.: 09/990,905

(22) Filed: Nov. 13, 2001

Related U.S. Application Data
(60) Provisional application No. 60/248,151, filed on Nov. 13, 2000.

(51) Int. Cl. A61F 13/00

(52) U.S. Cl. 602/62; 602/61

(57) ABSTRACT

An anchoring system for supporting an arm in a cast including Velcro™ formations on an elongated strap that depends around the neck of the wearer, the formations adjustably interact to support the arm at the appropriate level. Other components having Velcro™ formations can interact with the strap. The strap has releasable buckles or snaps along its length.

### BACKGROUND OF THE INVENTION

This invention relates to an arm support system. In particular, it relates to a system for supporting the wearer's arm. More specifically, when the arm is in a cast for medical purposes or must remain stationary for healing purposes (sprains, surgery, etc.).

Different sling systems have been used to support an arm in a cast. All currently existing sling support systems utilize a single strap, over the shoulder and side of the neck configuration. They suffer the disadvantages of creating undue pressure on the side of the shoulders and the neck. More so, they are unsightly and cumbersome and uncomfortable around the arm and the wrist, and require constant adjusting of the strap buckle and arm sleeve.

The invention is concerned with finding an easily usable device for supporting the arm, relieving shoulder and upper back strain and tension, preventing the sling height position from slipping, coming undone or sliding down from the arm support, and for permitting easy adjustment of the support height.

### SUMMARY OF THE INVENTION

By this invention there is provided an anchoring system for supporting a cast relative to the human torso.

In one form of the invention, around one or both of the shoulders or the neck of a wearer there depends a material having one or more hook formations. Around the arm of the wearer there is a mating one or more hook formations. These hooks can engage to support the arm at the appropriate level relative to the torso of the wearer.

In a form of the invention, there is an elongated strap configuration affixed around the neck like a depending tie or belt-like configuration for securing the strap and in turn the arm and cast. One or more inter-engaging buckle sets can be used in the length of the strap to ensure the effective and easy location around the neck. The free end of the strap or belt can loop about the cast and can, in turn, loop about itself to form a secure anchorage to prevent any slippage of the looped strap or belt downwardly due to the weight of the cast. Anchorage means may selectively be provided to secure the flap free end of the strap to a position removed from the free end of the strap. The anchorage means can selectively be a set of hook formations, particularly a set of Velcro™ hook and loop inter-engaging formations.

In another preferred form of the invention the support material, which is worn around the upper shoulders and both sides of the neck of the wearer, includes a Velcro™ face with multiple hook formations. The material around the cast includes mating hook formations of Velcro™. These hook formations can inter-engage so that the cast, namely the arm, is supported at the appropriate level and can be easily adjusted by the wearer to whatever height is intended.

In one preferred form of the invention, the material around the neck of the patient is in the form of a “tie” which is attached from the back of the neck with Velcro™, straps, buttons, zippers or other fastening devices. The front face of the tie has the Velcro™ formations. The formations around the cast can be in the form of a band with Velcro™ formations on the outside so as to engage the surface of the tie. By having the Velcro™ formations extend up the length of the tie for a determined length, the Velcro™ on the cuff can be arranged to engage the tie at different lengths, and thereby support the arm at different angular relationships relative to the torso, accommodating the different shape, weight and height of the wearer.

In another preferred form of the invention, the Velcro™ formations on the material around the body are formed on a face of a bib-like article which extends around the neck of the wearer. In yet a different form of the invention, the article in the form of a cravat or scarf-type configuration is attached around the neck as appropriate. In a different format, the elongated material can be wrapped around the neck and hooked together in a secured position back-to-back. In yet a different form of the invention, the material around the neck also extends over at least one or more shoulders to spread the load over the shoulders and minimize or eliminate the load around the neck.

The invention is described with reference to the accompanying drawings.

### BRIEF DESCRIPTION OF THE DRAWINGS

FIG. 1 is a representation of the upper body with an arm in a cast showing the tie configuration of the support means for the arm.

FIG. 2 is a cross-sectional representation showing the cast and a band around the cast with hooks.

FIG. 3 is an alternative representation of the cuff material for securing to the material extending down the body.

FIG. 4 is a front representation of a bib.

FIG. 5 is a representation of a cravat with the hook formation.

FIG. 6 is a representation of an elongated material wrapped around the neck and overlapping.

FIG. 7 is a representation of an extended shoulder support material extending over the front of the body with the hook material.

FIG. 8 is a cross-sectional view of the material over the shoulder.

FIG. 9 is a front view of a different embodiment of the invention having a single strap with buckles and selective holes in the strap or belt for securing the leading end of the strap to one or more buckles, and thereby supporting the cast.

FIG. 10 is a perspective-side view showing the buckle system of FIG. 9 secured around a cast.

FIG. 11 is a different view of a system using a buckle configuration without hooks, particularly without Velcro™ hooks.

### PREFERRED EMBODIMENT OF THE INVENTION

As illustrated in FIG. 1 there is a human upper torso which includes the chest region 10 and an arm 11 in a cast 12. The cast is shown extending over the elbow region 13. In some cases, the cast may extend over only the lower arm portion and not the upper arm portion. The hand and wrist 14 extend from the front end 15 of the cast. Around the neck 16 of the body there is a tie formation 17 which is knotted 18 in a conventional way similar to the knot of a tie worn for decoration. There is a portion 19 which embraces the neck from front to back, and an elongated portion 20 which extends down the front of the body 10 of the wearer. On the front face there are multiple hooks 21 which are formed by a Velcro™ pad material 22 which is attached, for instance, by gluing, securing etc. to the front base of the tie. This attachment can be by stitching or the like. In some cases, the tie as a whole can be made of a Velcro™ material over much of the length or the front portion of the length without there being a conventional tie backing material.

The mating portion of the Velcro™ (hook and loop) material is formed as a band 23 which extends over the cast 12. The band 23 may have overlapping edges 24 and 25 which engage each other so as to form a secure connection of the band. In other cases, the band may be formed on an elasticized-type material or of circumferentially secure material to fit over the cast 12. The outside face of the band is provided with multiple hooks 26 which are the mating Velcro™ hooks.

These hooks may extend totally around the band or just over that portion of the band, which would be part of the Velcro™ material 21. Alternatively, there can be rigid planar or flat material with the Velcro™ portion. In this fashion, the inter-engagement of the band 23 with the pad 21 in inter-engaging Velcro™ hook formation permits for the support of the arm relative to the upper body 10. As desired, the arm can be moved upwardly and downwardly relative to the torso and in this fashion the arm can be supported at the appropriate level relative to the torso. Since the material 17 for the tie in one form is of a flexible or loose nature, there may be sufficient movement provided to the arm in a manner similar to a sling, and even more so since the arm is not encumbered by a sling which passes around the neck.

In a different form of the invention, instead of a band 23, there can be a U-shaped cuff member 27 which has Velcro™ hook formations 28 on one face for engagement with the tie member 17. The hooking Velcro™ material 28 attaches to the sling.

In yet another form of the invention, instead of a tie formation there can be a bib structure 29 which can lay over the front of the person needing the arm support to wrap formations 30 and 31 extend to the back of the neck. A hook configuration 32 and 33 on mating opposite ends of the straps can be used for securing the bib around the body utilizing attaching material such as Velcro™, buttons, snaps, zippers, etc. The Velcro™ elements 34 can be provided on the front face of the bib over any part of the bib that may be required. Thus, in some cases, instead of being vertically centrally located down the bib, the Velcro™ formations can be located on other portions of the bib 29, for instance on the side portions of the bib.

As shown in FIG. 5 there is a cravat-type configuration 35. The advantage of the cravat is a broader band 36 which extends around the neck. This has the advantage of distributing the pressure of the support around a greater portion of the neck and upper back area. It is therefore different to a tie configuration which has a generally narrower profile. The cravat or scarf 35 can be appropriately affixed by folding, knotting or clipping down the front of the body as required.

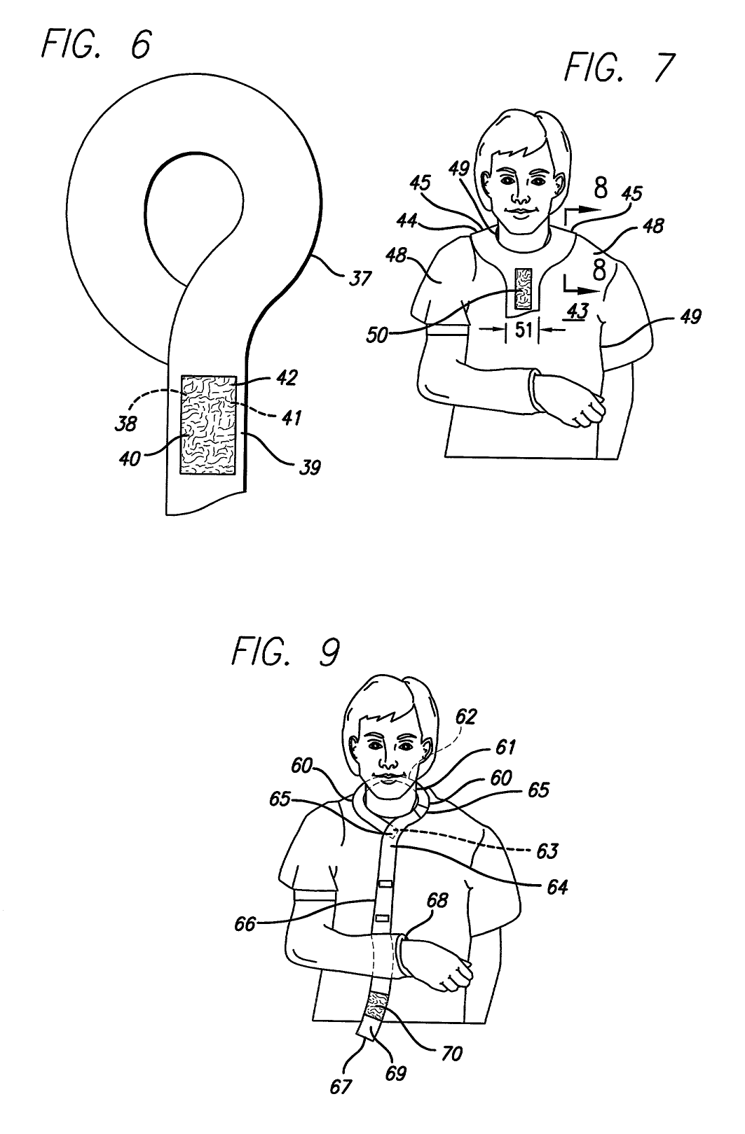

In the embodiment shown in FIG. 6 there is an elongated material 37 which wraps around the neck. The back portion 38 extends behind the front face 39 of the elongated material. Suitable hook formations 40 on the back face of the front portion 38 and hook formations 41 on the front face of the rear portion 38 are used in inter-engage and form a secure structure around the neck. These hook formations 40 and 41 can be suitable Velcro™ material in the mating relationship. The hook formations 42 on the front face 39 are provided for securing the cuff, band or other support which exists around the cast. Although the cuff or band is shown in FIG. 1 as being formed around the cast directly, there are situations where, when a shirt is worn by the user, the cuff is located over the shirt, jacket or the like sleeve of the user.

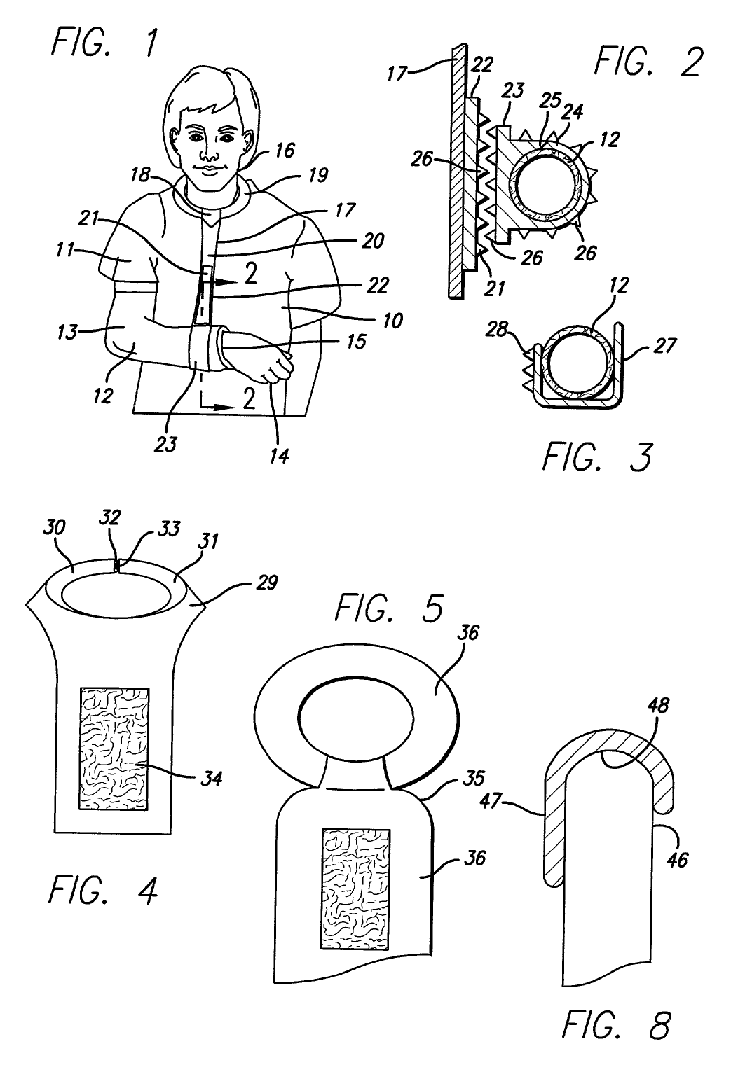

In a variation of the embodiment of FIG. 6, when the wrap around portion is placed about the neck there is a buckle or snap towards the end of the free end which engages matingly with a mating buckle or snap portion on the elongated downwardly depending front portion 39. This secures the wrap around about the neck. The front face of hooks 40 can extend over an extended length down the front face 39 of the elongated strap 37. A separate strap or band can be formed for embracing he cast as shown in FIG. 2. The separate band can have formations of hooks and/or loops of Velcro™ material formed on the inner and/or outer face of the separate band or strap. As such the separate band or strap forms a cuff for surrounding the cast, and the inter-engagement of the hooks and loops at the overlapping edges 24 and 25 can form a tighter or looser cuff around the cast as required. A free end or surface of the separate band is also provided with the mating hooks or loops to engage the respective hooks 40 on the front fac39 in the manner illustrated in FIG. 2. As such the cuff formed by the separate strap or band can be located at an appropriate and adjustable level or height as required.

In FIGS. 7 and 8 there is yet a further embodiment of the invention which is illustrated. Here, a T-shaped type of garment 43 is provided with shoulder extending portions 44 and 45, respectively. The shoulder extending portions extend downwardly at 46 behind the wearer and 47 in front of the wearer over the shoulder region 48. The advantage of this is that the support for the arm is extended over the shoulders 48 and not simply around the neck portion 49 of the wearer. The material in this case may be more rigid than a simple flexible material so that the force and pressure is extended over the width of the material from shoulder towards the other shoulder. Suitable hook formations 49 are provided in the front face of the material to engage the cuff. In this case, the front face 50 of the material does not extend as a broad bib over the front of the body that is more narrowly defined between a broad bib and a tie-type configuration. The width 51 can be determined appropriately so that the weight is distributed in an appropriate manner.

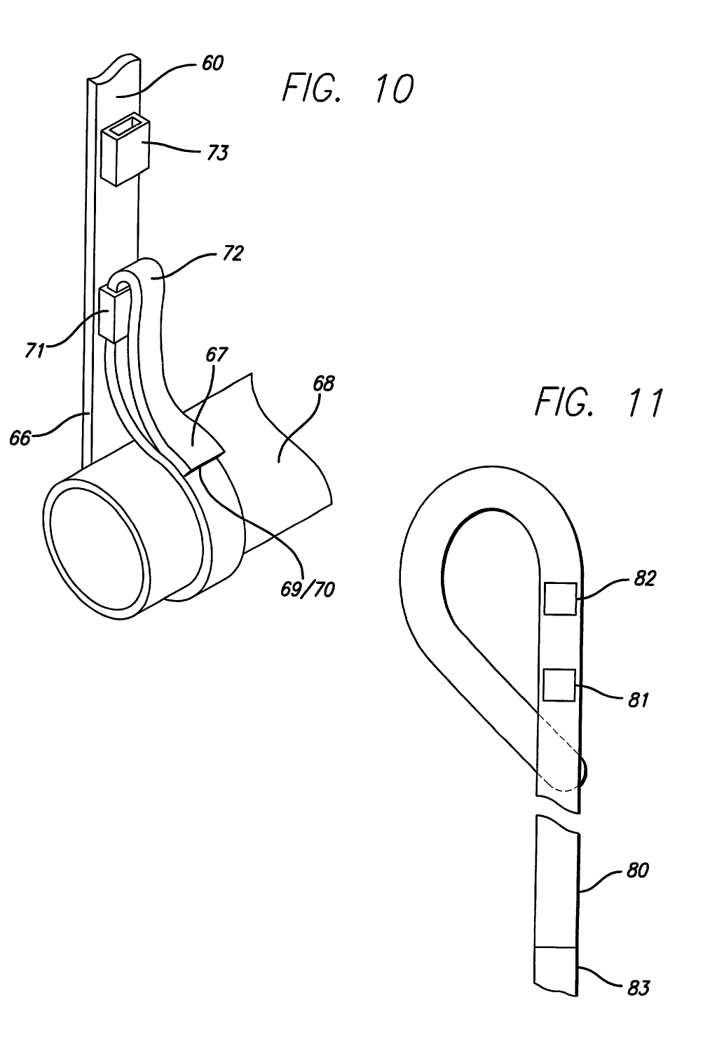

As illustrated in FIG. 9 there is an elongated strap 60 which fits around the neck 61 of a wearer as shown by dotted line 62. One free end 63 of the strap is attached to an opposite portion 64 of the strap by an appropriate means which can be stitching, Velcro™ hooks or other affixation means at the zone indicated by numeral 65. This can be one or more releasable buckle or clip sets which can be arranged at one or more locations along the length of the strap. The inter-engagement and disengagement can be effected with a relatively easy snap action operable with relatively light finger pressure by a wearer. Ideally there are at least two buckle sets 65 which are spaced apart on the elongated component lengths of the strap. As such the elongated length can be made of at least three separate elongated components. Alternatively, the anchorage at 65 can be a permanent relationship created by sewing or the like.

The strap 66 then depends downwardly down the front portion of the torso, and has a free end 67. The arm and cast 68 pass through the strap at an appropriate level. The end portion 67 has one face 69 with one form of Velcro™ hook, and the portion 70 slightly removed from the end portion 70 has an opposite side of the Velcro™. Thus, if 69 and 70 engage with each other, they are mated in a hook and loop formation created by the Velcro™ configuration.

In use, the cast 68 is placed adjacent to the length 66 and the free end 67 is looped upwardly and over the cast 68 to engage a first buckle 71. The free end 67 passes through the buckle 71 and is looped at 72 such that the portion 69 and 70 inter-engage. In this sense, the Velcro™ on the back side 69 engages with the Velcro™ on the front side 70 as generally shown in FIG. 10. The Velcro™ 70 may extend upwardly to any desired degree along the strap 66. There is an additional buckle 73 which is provided at a higher length closer to the portion 65. When the belt is looped through buckle 73 as opposed to 71, the cast 68 is held at a higher level. This looping of the strap and inter-engagement of the free end through a hook formation provides an effective positive securing mechanism for the cast.

In different forms of the invention, the thickness of the belt 60 around the neck 61 as indicated by 62 can be varied so as to provide an appropriate comfort to the neck and shoulders. Suitable cushioning can be provided to that portion of the configuration.

As shown in FIG. 11 there is an elongated strap 80. The bottom portion is not shown but can be similar to the configuration illustrated in FIG. 7. The depending portion may have one or more apertures 81 or 82 formed in the length, and the free end 83 can pass through those apertures. The apertures 81 and 82 engage with the free end 83 can include one or more buckles, snaps or clip sets for permitting a locking attachment mechanism so as to provide a secure loop for supporting a cast 68. Instead of buckles and apertures 81 and/or 82, there could be hook and eye or other securing mechanisms. This belt-type configuration can provide effective securing of the cast. Similarly, the structure around the neck is simplified and constructed so as to provide effective dispersion of the pressure around the neck and shoulders. In the embodiment shown in FIGS. 9, 10 and 11, the outside surface of the cast 68 may have, as necessary, Velcro™ pads to facilitate a mating of Velcro™ hook and loop formations on one or other of the surfaces of the Velcro™ strap. This would add provided security and anchorage for the cast.

Many other forms of the invention exist, each differing from the other in matter of detail only.

In some cases only a single row or line of hooks may be provided to extend up the length of the tie, cravat or elongated material. In other words, instead of a Velcro™ material, a different hook formation may be provided to create the secure anchorage necessary for the cast. For instance, the hook and eye formations used in female brassieres. Also, the formation around the cast may only have one or a limited number of hooks to engage the hooks extending vertically up the elongated material in front of the torso.

Various other forms of the invention exist. Different embodiments differ in detail only from the concept of the invention as described. The invented system may have particular benefits to women due to their anatomy.

What is claimed is:

1. An anchoring system for supporting a cast about an arm relative to the human torso comprising:
an elongated strap material having first hook formations located along the length of the strap, the elongated strap depending around one or both of the shoulders or the neck of a wearer; and
one or more second hook formations for mating with the first hook formations on the strap and being connectable in a releasable manner with the first hook formations, the second hook formations being located around the arm of the wearer thereby to support the arm at the appropriate level relative to the torso of the wearer.

2. An anchoring system as claimed in claim 1 wherein the elongated strap material is locatable about the shoulders or the neck of the wearer, and the strap includes one or more releasable buckle formations to separate or connect portions of the strap to form the elongated length for securing around the neck or shoulders.

3. An anchoring system as claimed in claim 1 wherein the elongated strap material extends around the neck and over at least one or more shoulders thereby to spread the load over the shoulders and minimize or eliminate the load around the neck.

4. An anchoring system for supporting an arm relative to the human torso comprising:
an elongated strap material having one or more first hook formations for depending around one or both of the shoulders or the neck of a wearer;
one or more second hook formations for mating with the first hook formations to secure the strap about the shoulders or the neck of the wearer, hook formations being located around the arm of the wearer to be
connected with the strap thereby to support the arm at the appropriate level relative to the torso of the wearer; and
wherein the hook formations include a set of Velcro™ hook formations, on the strap for securing the arm at an adjustable level.

5. An anchoring system as claimed in claim 4 wherein the arm is located in a cast, and wherein a free end of the strap loops about the arm and, in turn, loops about itself to form a secure anchorage to minimize slippage of the looped strap downwardly due to the weight of the cast.

6. An anchoring system as claimed in claim 5 including hooks for securing the flap free end of the strap to a position removed from the free end of the strap.

7. An anchoring system as claimed in claim 1 including support material to be worn around the upper shoulders and both sides of the neck of the wearer, the support material including a Velcro™ face with multiple hook formations.

8. An anchoring system as claimed in claim 1 including material around a cast worn over the arm, mating hook formations of Velcro™ on the material, the hook formations inter-engaging with Velcro™ hook formation on the strap so that the cast and the arm is supported at an appropriate level and the support being adjustable by the wearer to a selected height.

9. An anchoring system as claimed in claim 1 wherein the strap material around the neck is in the form of a neck tie, the material being attached at the back of the neck with a fastener selected from the group of Velcro™, strap, button, or a zipper.

10. An anchoring system as claimed in claim 9 wherein a front face of the tie includes Velcro™ hook formations, and including mating Velcro™ formations around a cast selectively in the form of a band thereby to engage the Velcro™ hook formation on the surface of the tie.

11. An anchoring system as claimed in claim 10 wherein the Velcro™ formations extend up the length of the tie for a determined length, and the Velcro™ around the cast are arranged to engage the tie at different lengths, and thereby support the arm at different angular relationships relative to the torso thereby accommodating the different shape, weight and height of the wearer.

12. An anchoring system as claimed in claim 1, wherein the strap material includes a face of a bib-like article, the strap for the bib extending around the neck of the wearer.

13. An anchoring system as claimed in claim 1, wherein the strap is in the form of a cravat or scarf-type configuration located around the neck.

14. An anchoring system for supporting a cast about an arm relative to the human torso comprising:
an elongated strap material for depending around one or both of the shoulders or the neck of a wearer, the strap having opposite surfaces;
Velcro™ formations along one or both of the opposite surfaces, the formations not being around the portion of the strap about the neck or shoulders; and
an end of the strap being located about a cast and being connected with the Velcro™ hook formations thereby to secure the cast, and the location of the Velcro™ formation connection being variably determined along the length of the strap such that the location of support of the arm relative to the torso is variable.

15. An anchoring system as claimed in claim 14 including a set of hook formations, with the strap thereby to releasably affix the strap around the neck.

16. An anchoring system as claimed in claim 14 wherein the elongated strap material is locatable about the shoulders or the neck of the wearer, and the strap includes one or more releasable buckle formations to separate or connect portions of the strap to form the elongated length for securing around the neck or shoulders.

17. An anchoring system as claimed in claim 14 wherein the free end of the strap loops about the cast and, in turn, loops about itself to form a secure anchorage to minimize slippage of the looped strap downwardly due to the weight of the cast.

18. An anchoring system as claimed in claim 14 including hooks provided to secure the flap free end of the strap to a position removed from the free end of the strap.

19. An anchoring system for supporting a cast about an arm relative to the human torso comprising:
an elongated strap material for depending around one or both of the shoulders or the neck of a wearer, the strap having opposite surfaces;
one or more releasable buckle formations to separate or connect portions of the strap to form the elongated length for securing around the neck or shoulders;
Velcro™ formations along the front surface of the strap;
a separate band having Velcro™ hook or loop formations for connection with the Velcro™ hook or loop formations on the front surface; and
the separate band of Velcro™ hook or loop formations forming a closable cuff about the cast, and the location of the Velcro™ formations extending for a predetermined length along the front surface of the strap such that the location of cuff support with inter-engaging Velcro™ hook or loop formations is variably locatable with the front surface and the arm is thereby is variably locatable relative to the torso.
